(19) United States
(12) Patent Application Publication
(10) Pub. No.: US 2026/0083782 A1
(43) Pub. Date: Mar. 26, 2026

(54) SELECTIVE ANTIMICROBIAL USE OF MICROCOCCUS PORCI

(71) Applicant: CutisBio Co., Ltd., Seoul (KR)

(72) Inventors: Won Woo CHOI, Seoul (KR); Byongjun CHOI, Seoul (KR); Minji PARK, Gyeonggi-do (KR); Wonduck KIM, Gyeonggi-do (KR)

(21) Appl. No.: 18/893,173

(22) Filed: Sep. 23, 2024

(51) Int. Cl. A61K 35/74; C12N 1/20; C12R 1/265

(52) U.S. Cl. CPC A61K 35/74; C12N 1/205; C12R 2001/265

(57) ABSTRACT

The present invention relates to the selective antimicrobial use of Micrococcus porci against harmful Staphylococcus sp. A composition comprising Micrococcus porci, or a lysate, culture or extract thereof, as an active ingredient, exhibits selective antimicrobial activity against harmful Staphylococcus sp., in particular, Staphylococcus aureus, Methicillin resistance Staphylococcus aureus (MRSA), and Staphylococcus capitis, and thus can be used as antimicrobial compositions in various field such as cosmetics, quasi-drugs, external skin preparations, or medicines.

### SEQUENCE LISTING

This application contains references to amino acid sequences and/or nucleic acid sequences which have been submitted concurrently herewith as the sequence listing XML file entitled “000002 us_SequenceListing.XML”, file size 4,856 bytes, created on 23 Sep. 2024. The aforementioned sequence listing is hereby incorporated by reference in its entirety pursuant to 37 C.F.R. § 1.52 (e) (5).

### TECHNICAL FIELD

The present invention relates to a selective antimicrobial use of Micrococcus porci against harmful Staphylococcus sp. More specifically, the present invention relates to a selective antimicrobial use of a composition against harmful Staphylococcus sp., which comprises Micrococcus porci, or a lysate, culture or extract thereof as active ingredient.

### BACKGROUND

Since the surface of human skin contains a lot of waste products such as dead skin cells, sweat, and sebum, various microorganisms live on the skin and form the skin microbiome, which is the skin flora. Beneficial and harmful bacteria live in symbiosis with each other in the skin flora, and the distribution of beneficial bacteria is larger in healthy skin, while the distribution of harmful bacteria is larger in atopic or acne-prone skin. Therefore, it is known that maintaining the balance of the skin flora helps to maintain a healthy skin condition.

The skin's normal resident bacteria form a skin barrier that prevents the penetration of other more harmful microorganisms (pathogens) and contribute to the skin's immune system. An imbalance between the skin's beneficial and harmful bacteria can lead to a breakdown in the skin barrier function and a decrease in the skin's immune response, which may cause various skin conditions such as atopic dermatitis, seborrheic dermatitis, acne, and psoriasis. Therefore, selective inhibition of the activity of harmful bacteria without affecting the skin's beneficial bacteria would be beneficial in treating various skin diseases.

In the present invention, studies on skin commensal bacteria have confirmed that Micrococcus porci exhibits selective antimicrobial activity against harmful Staphylococcus sp.

### DESCRIPTION OF THE INVENTION

### Problems to be Solved

Accordingly, an object of the present invention is to provide an antimicrobial cosmetic composition comprising as an active ingredient Micrococcus porci, or a lysate, culture or extract thereof, which exhibits selective antimicrobial activity against harmful Staphylococcus sp.

Another object of the present invention is to provide an antimicrobial quasi-drug composition comprising as an active ingredient Micrococcus porci, or a lysate, culture or extract thereof, which exhibits selective antimicrobial activity against harmful Staphylococcus sp.

Another object of the present invention is to provide an antimicrobial external skin preparation as an active ingredient Micrococcus porci, or a lysate, culture or extract thereof, which exhibits selective antimicrobial activity against harmful Staphylococcus sp.

Another object of the present invention is to provide an antimicrobial health food composition comprising as an active ingredient Micrococcus porci, or a lysate, culture or extract thereof, which exhibits selective antimicrobial activity against harmful Staphylococcus sp.

Another object of the present invention is to provide an antimicrobial pharmaceutical composition comprising as an active ingredient Micrococcus porci, or a lysate, culture or extract thereof, which exhibits selective antimicrobial activity against harmful Staphylococcus sp.

In addition, another object of the present invention is to provide a method of treating, ameliorating, or alleviating a harmful Staphylococcus sp. infection or a condition caused thereby, comprising administering to a subject in need thereof an antimicrobial composition comprising Micrococcus porci, or a lysate, culture or extract thereof.

Another object of the present invention is to provide a method of inhibiting the growth of harmful Staphylococcus sp., comprising applying an antimicrobial composition comprising Micrococcus porci, or a lysate, culture or extract thereof, to an area where the growth of harmful Staphylococcus sp. is suspected.

Furthermore, still another object of the present invention is to provide a strain of Micrococcus porci deposited under Accession No. KACC 81219BP.

Another object of the present invention also is to provide a lysate, culture or extract of said Micrococcus porci strain.

### Technical Solution

One aspect of the present invention provides an antimicrobial cosmetic composition comprising as an active ingredient Micrococcus porci, or a lysate, culture or extract thereof, which exhibits selective antimicrobial activity against harmful Staphylococcus sp.

In the present invention, Micrococcus porci may be either live (live bacteria) or dead (dead bacteria). More specifically, said dead bacteria may be heat-treated dead bacteria.

In one embodiment, Micrococcus porci may be the Micrococcus porci strain deposited under Accession No. KACC 81219BP.

As used herein, the term “lysate” refers to a solution or suspension in an aqueous medium of cells of a microorganism, such as broken Micrococcus porci. The cell lysate comprises, for example, macromolecules such as DNA, RNA, proteins, peptides, carbohydrates, lipids, etc. and/or small molecules such as amino acids, sugars, fatty acids, etc. or fractions thereof. Furthermore, said lysate comprises cellular debris, which may be smooth or granular in structure.

A variety of known methods can be used to achieve cell lysis of the microorganism, and any method capable of achieving cell lysis of the microorganism can be used. For example, cell opening or disruption may be performed enzymatically, chemically, or physically. Non-limiting examples of enzymes and enzyme mixtures are proteases, such as protease K, lipases, or glycosidases, and non-limiting examples of chemicals are ion-permeable carriers, detergents such as sodium dodecyl sulfate, acids, or bases. Non-limiting examples of physical means are high pressure, such as French pressing, osmotic pressure, and temperature, such as heat or cold. Additionally, methods using any suitable combination of enzymes, acids, bases, etc. other than proteolytic enzymes may also be used.

As used herein, the term “culture” may be used interchangeably with “culture supernatant”, “conditioned culture” or “conditioned medium”, and may refer to the entire medium comprising Micrococcus porci, its metabolites, extra nutrients, etc. obtained by culturing Micrococcus porci for a period of time in a medium capable of providing nutrients to enable Micrococcus porci to grow and survive in vitro. Furthermore, said culture may refer to a culture medium obtained by removing the bacterial cells from the bacterial culture medium obtained by culturing the microorganism. The liquid from which the bacterial cells have been removed from the culture medium is also referred to as the “supernatant”, which can be obtained by allowing the culture to stand still for a certain period of time and taking only the liquid in the upper layer excluding the part settled in the lower layer, removing the bacterial cells by filtration, or centrifuging the culture to remove the sedimentation of the lower layer and taking only the liquid in the upper layer. The “bacterial cells” refer to the microorganism itself of the present invention, and include the microorganism isolated and screened from a skin sample or the like, or the microorganism isolated from a culture medium by culturing said microorganism. The bacterial cells can be obtained by centrifuging the culture and taking the portion that has settled to the bottom layer, or by allowing the bacterial cells to settle to the bottom layer of the culture by gravity, and then removing the liquid from the top layer after allowing the bacterial cells to stand still for a period of time.

According to one aspect, the culture of Micrococcus porci of the present invention may use any medium readily selected by a person of ordinary skill in the art according to the intended purpose from among those used for microbial culture. Specifically, a medium used for micrococcus culture, such as Mueller Hinton (MH) broth or Tryptic soy broth (TSB) broth can be used, but is not limited thereto. According to one embodiment, the culture of Micrococcus porci of the present invention can be prepared by inoculating Micrococcus porci into said microbial culture medium and using any microbial culture method known in the art (e.g., stationary culture, etc.).

Said culture may comprise the culture medium itself obtained by culturing the microorganism, or a concentrate or lyophilized product thereof, or a culture supernatant obtained by removing the microorganism from the culture medium, or a concentrate or lyophilized product thereof.

The culture may be obtained by culturing Micrococcus porci in a suitable medium (e.g., MH medium) at any temperature between 10° C. and 40° C. for a period of time, for example, 4 to 50 hours.

In one embodiment, the culture supernatant of the microorganism may be obtained by centrifuging or filtering the microbial culture to remove the microorganism.

In another embodiment, the concentrate may be obtained by the step of concentrating said microbial culture itself, or the supernatant obtained after filtering said culture using a centrifuge or filter.

Culture media and culture conditions for culturing the Micrococcus porci may be appropriately selected or modified by one of ordinary skill in the art.

As used herein, the term “extract” means an extract from said lysate, culture, or concentrate thereof, and may include an extract, a diluent or concentrate of an extract, a dry product obtained by drying an extract, or an adjustment or purification thereof, or fractions thereof.

The composition may comprise from 0.00001% to 80% by weight, for example, 0.00001% to 60% by weight, 0.00001% to 40% by weight, 0.00001% to 30% by weight, 0.00001% to 20% by weight, 0.00001% to 10% by weight, 0.00001% to 5% by weight, 0.05% to 60% by weight, 0.05% to 40% by weight, 0.05% to 30% by weight, 0.05% to 20% by weight, 0.05% to 10% by weight, 0.05% to 5% by weight, 0.1% to 60% by weight, 0.1% to 40% by weight, 0.1% to 30% by weight, 0.1% to 20% by weight, 0.1% to 10% by weight, or 0.1% to 5% by weight of the microorganism, or a lysate, culture or extract thereof, based on the total weight of the composition.

As used herein, the term “comprising as an active ingredient” means that the microorganism, or a lysate, culture or extract thereof is added in an amount sufficient to produce the effects described above, and includes formulations in various forms with various ingredients added as excipients for drug delivery, stabilization, etc.

As used herein, the term “antimicrobial” means having the activity of (i) inhibiting, reducing or preventing the growth of bacteria; (ii) inhibiting or reducing the ability of bacteria to cause infection in a subject; or (iii) inhibiting or reducing the ability of bacteria to multiply or remain infectious in the environment.

In the present invention, Micrococcus porci, or a lysate, culture or extract thereof, may selectively show antimicrobial activity against harmful Staphylococcus sp. Specifically, Micrococcus porci exhibits antimicrobial activity selectively against harmful Staphylococcus sp., while having weak or no effect on harmless skin beneficial bacteria.

The term harmful Staphylococcus sp. means a staphylococcus that penetrates the skin surface or tissue of a host and causes skin rashes, infections (abscesses, pus, redness, etc.), and other problems. Said Micrococcus porci, or a lysate, culture or extract thereof may exhibit antimicrobial activity against harmful staphylococci of the genus Staphylococcus, such as, but not limited to, a skin disease-causing bacteria Staphylococcus aureus, Methicillin resistance Staphylococcus aureus (MRSA), and Staphylococcus capitis. However, it has been found that Micrococcus porci, or a lysate, culture or extract thereof has weak antimicrobial activity against Staphylococcus epidermidis, a skin-beneficial bacteria, and has almost no antimicrobial activity against Staphylococcus hominis, a skin-beneficial bacteria. Furthermore, Micrococcus porci, or a lysate, culture or extract thereof has been found to exhibit little antimicrobial activity against Micrococcus luteus, Candida albicans SC5314, Corynebacterium propinguum, Pseudomonas aeruginosa ATCC10145, and Escherichia coli DH5a, which do not belong to Staphylococcus sp.

In one embodiment, said Micrococcus porci, or a lysate, culture or extract thereof can exhibit selective antimicrobial activity against at least one of Staphylococcus aureus, Methicillin resistance Staphylococcus aureus (MRSA), and Staphylococcus capitis.

In one embodiment, said Micrococcus porci, or a lysate, culture or extract thereof can exhibit selective antimicrobial activity against Staphylococcus aureus, Methicillin resistance Staphylococcus aureus (MRSA), and Staphylococcus capitis.

In the present invention, a cosmetic composition may be prepared in a formulation selected from the group consisting of a cosmetic water (skin lotion), skin, skin softener, skin toner, astringent, lotion, milk lotion, moisturizing lotion, nourishing lotion, massage cream, nourishing cream, moisturizing cream, hand cream, hand sanitizer, foundations, essences, nutritional essences, packs, soaps, cleansing foams, cleansing lotions, cleansing creams, body lotions, body cleansers, suspensions, gels, powders, pastes, masks, and sheets. The compositions of these formulations can be prepared according to methods conventionally known in the art. The amounts of additional ingredients, such as moisturizers, can be readily selected by one of ordinary skill in the art without compromising the object and effectiveness of the present invention.

The cosmetic composition may further comprise, in addition to the active ingredients disclosed herein, functional additives and ingredients commonly included in cosmetic compositions, and may further comprise conventionally used purified water, thickeners, preservatives, stabilizers, solubilizers, surfactants, carriers, fragrances, or combinations thereof. Said functional additives may include ingredients selected from the group consisting of water-soluble vitamins, useful vitamins, polymeric peptides, polymeric polysaccharides, sphingolipids, and seaweed extracts. The carriers may include, for example, alcohols, oils, surfactants, fatty acids, silicone oils, wetting agents, humectants, viscosity modifiers, emulsions, stabilizers, UV scatterers, UV absorbers, colorants, fragrances, and the like. The compounds or compositions that can be used as alcohols, oils, surfactants, fatty acids, silicone oils, humectants, moisturizers, viscosity modifiers, emulsions, stabilizers, UV scatterers, UV absorbers, colorants, fragrances, etc. are well known in the art and those skilled in the art will be able to select and use any suitable substance or composition. The cosmetic composition may also contain sunscreens, antioxidants (such as butylhydroxyanisole, propyl gallate, erythorbic acid, tocopheryl acetate, butylated hydroxytoluene, etc.), preservatives (such as methylparaben, butylparaben, propylparaben, phenoxyethanol, imidazolidinyl urea, chlorphenesin, etc.), colorants, pH adjusters (triethanolamine, citric acid, sodium citrate, malic acid, sodium malate, fumaric acid, sodium fumarate, succinic acid, sodium succinate, sodium hydroxide, sodium monohydrogen phosphate, etc.), moisturizers (glycerin, sorbitol, propylene glycol, butylene glycol, hexylene glycol, diglycerin, betaine, glycereth-26, methylglucose-20, etc.), lubricants, etc., as necessary.

Furthermore, in the cosmetic composition of each formulation, appropriate ingredients can be selected and formulated according to the formulation or intended use of the cosmetic. Since the ingredients and methods of formulation can be determined in accordance with conventional techniques, detailed descriptions thereof are omitted herein.

Another aspect of the present invention provides an antimicrobial quasi-drug composition comprising, as an active ingredient, Micrococcus porci, or a lysate, culture or extract thereof, which exhibits selective antimicrobial activity against harmful Staphylococcus sp.

The Micrococcus porci, or a lysate, culture or extract thereof, and their selective antimicrobial activity against harmful Staphylococcus sp. are the same as described above.

As used herein, the term “quasi-drug” refers to articles used for the purpose of diagnosing, treating, ameliorating, alleviating, curing, treating or preventing diseases of humans or animals that are milder in action than drugs. For example, according to the Pharmaceutical Affairs Act, quasi-drugs exclude articles used for medicinal purposes, and include textile and rubber products used for the treatment or prevention of diseases in humans and animals, those that have a minor or no direct effect on the human body, non-instrumental or mechanical devices and similar products, sterilizers and pesticides to prevent infectious diseases, etc.

The types or formulations of the quasi-drug composition of the present invention are not particularly limited, but may include bandages, gauze, cotton wool, band-aids, antiseptic cleaners, shower foams, gargles, wipes, detergent soaps, hand washes, humidifier fillers, masks, or filter fillers.

When the composition of the present invention is used as a quasi-drug product for selective antimicrobial use, the composition may be used as is or in combination with other quasi-drug product ingredients, and may be appropriately used according to conventional methods. The amount of active ingredients can be appropriately determined according to the intended use. The quasi-drug composition according to the present invention may comprise from 0.01% to 20% by weight of the microorganism, or lysate, culture or extract thereof, based on the total weight of the composition.

In another aspect, the present invention provides an antimicrobial external skin preparation, as an active ingredient, Micrococcus porci, or a lysate, culture or extract thereof, which exhibits selective antimicrobial activity against harmful Staphylococcus sp.

The Micrococcus porci, or a lysate, culture or extract thereof, and their selective antimicrobial activity against harmful Staphylococcus sp. are the same as described above.

The external skin preparation may be in the form of a cream, gel, ointment, skin emulsion, skin suspension, transdermal patch, lotion, or any combination thereof. The external skin preparation may be appropriately mixed with ingredients conventionally used in external skin preparations for cosmetics and pharmaceuticals, such as water-based ingredients, oil-based ingredients, powder ingredients, alcohols, moisturizers, thickeners, ultraviolet absorbers, whitening agents, preservatives, antioxidants, surfactants, fragrances, colorants, various types of skin nutrients, or a combination thereof, as necessary. The external skin preparation may be suitably formulated with metal sequestering agents such as sodium edetate, trisodium edetate, sodium citrate, sodium polyphosphate, sodium metaphosphate and gluconic acid, drugs such as caffeine, tannin, verapamil, licorice extract, glabridin, hot water extract of fruits of carlin, various types of herbal medicines, tocopherol acetate, glycyrrhizinic acid, tranexamic acid and derivatives or salts thereof, vitamin C, magnesium ascorbyl phosphate, ascorbic acid glucoside, albutin, kojic acid, and sugars such as glucose, fructose or trehalose.

The external skin preparation according to the invention may comprise from 0.00001% to 80% by weight of the microorganism, or a lysate, culture or extract thereof, based on the total weight of the composition.

The skin includes any skin area of the body, including the face, hands, arms, legs, feet, chest, stomach, back, buttocks, and scalp.

In still another aspect, the present invention provides an antimicrobial health food composition comprising, as an active ingredient, Micrococcus porci, or a lysate, culture or extract thereof, which exhibits selective antimicrobial activity against harmful Staphylococcus sp.

The Micrococcus porci, or a lysate, culture or extract thereof, and their selective antimicrobial activity against harmful Staphylococcus sp. are the same as described above.

The health food composition may comprise Micrococcus porci or its lysates, cultures, or extracts alone or in combination with other foods or food ingredients, and may be used as appropriate according to conventional methods. The amount of the active ingredient in the mixture may be suitably determined according to the intended use (preventive, health or therapeutic treatment). Generally, the composition of the present disclosure may be added in an amount of not more than 15 parts by weight to the raw material in the preparation of a food or beverage.

There is no particular restriction on the type of health food. Examples of food products to which the composition may be added include formulations selected from the group consisting of acids, granules, tablets, capsules, pills, gels, jellies, suspensions, emulsions, syrups, tea bags, leached teas, and health beverages, all of which are health foods in the conventional sense. The health beverage composition among the health foods may additionally include various flavors, natural carbohydrates, etc., like other beverages.

The natural carbohydrates may include monosaccharides such as glucose and fructose, disaccharides such as maltose and sucrose, polysaccharides such as dextrin and cyclodextrin, and sugar alcohols such as xylitol, sorbitol and erythritol. Natural sweeteners such as thaumatin, stevia extract, or synthetic sweeteners such as saccharin, aspartame, etc. can be used as sweeteners. The health food composition may also contain nutrients, vitamins, electrolytes, flavors, colorants, pectic acid and salts thereof, alginic acid and salts thereof, organic acids, protective colloidal thickeners, pH adjusters, stabilizers, preservatives, glycerin, alcohols, carbonation agents used in carbonated beverages, or combinations thereof. The health food composition may also contain a natural fruit juice, a fruit juice beverage, a pulp for the preparation of a vegetable beverage, or a combination thereof.

In still another aspect, the present invention provides an antimicrobial pharmaceutical composition comprising Micrococcus porci, or a lysate, culture or extract thereof, as an active ingredient, which exhibits selective antimicrobial activity against harmful Staphylococcus sp.

The Micrococcus porci, or a lysate, culture or extract thereof, and their selective antimicrobial activity against harmful Staphylococcus sp. are the same as described above.

As used herein, the term “pharmaceutical composition” may refer to a molecule or compound that confers some beneficial effects upon administration to a subject. Advantageous effects may include: enabling diagnostic decisions; ameliorating a disease, symptom, disorder, or condition; reducing or preventing the onset of a disease, symptom, disorder, or condition; and generally, responding to a disease, symptom, disorder, or condition.

As used herein, the term “prevention” refers to actions taken to partially or completely delay or prevent the onset or recurrence of a disease, disorder, or concomitant symptom thereof, to prevent the acquisition or reacquisition of a disease or disorder, or to reduce the risk of acquiring a disease or disorder. For example, said prevention means all of actions that suppress or delay the occurrence of skin damage or symptoms by administration of the composition according to the present invention.

As used herein, the term “treatment” includes inhibiting, alleviating or eliminating the development of a disease.

As used herein, the term “ameliorating” refers to all of actions that result in the alleviation of a condition or at least a reduction in a parameter associated with treatment, such as the severity of a symptom.

In the pharmaceutical composition according to the present invention, Micrococcus porci, or a lysate, culture or extract thereof can be included as an active ingredient in a pharmaceutical composition having antimicrobial activity, or can be used as an adjuvant to an active ingredient having medicinal use. The pharmaceutical composition can be prepared according to methods well known to a person of ordinary skill in the art.

The pharmaceutical composition may be administered orally or parenterally, depending on the intended method, and may be used in the form of a conventional pharmaceutical formulation. Parenteral administration may refer to administration through a route, such as rectal, intravenous, peritoneal, intramuscular, arterial, transdermal, nasal, inhale, ocular, and subcutaneous administration, other than oral administration. When the pharmaceutical composition of the present invention is used as a medicine, it may further comprise one or more active ingredients exhibiting the same or similar function.

The pharmaceutical composition may further comprise one or more pharmaceutically acceptable carriers. Examples of the pharmaceutically acceptable carrier include saline, sterile water, Ringer's solution, buffered saline, dextrose solution, maltodextrin solution, glycerol, and ethanol. They may be used alone or in combination. If necessary, the pharmaceutical composition may further comprise other typical additives such as antioxidants, buffers, bacteriostatic agents, etc. The pharmaceutical composition may be formulated with a diluent, a dispersant, a surfactant, a binder and a lubricant into an injection dosage form such as aqueous solution, suspension, emulsion, etc., pill, capsule, granule, or tablet. Furthermore, the pharmaceutical composition can be formulated by any suitable method in the art, preferably according to the respective disease or according to the ingredients.

The pharmaceutical composition may be formulated using a diluent or excipient, such as a filler, an extender, a binder, a humectant, a disintegrant, a surfactant, etc. Formulations for parenteral administration may include sterilized aqueous solutions, non-aqueous solvents, suspensions, emulsions, lyophilized formulations, and suppositories. Examples of the non-aqueous solvents and suspensions may include propylene glycol, polyethylene glycol, and vegetable oils such as olive oil, an injectable ester such as ethyl oleate, etc. Examples of the bases for suppositories may include Witepsol, macrogol, Tween 61, cacao butter, laurinum, glycerogelatin, etc.

The pharmaceutical composition may be formulated with carbohydrates such as glucose, sucrose or dextran, antioxidants such as ascorbic acid or glutathione, chelating agents, small molecular proteins, or other stabilizers to increase stability or absorbability.

Another aspect of the present invention provides a method of preventing, treating, ameliorating, or alleviating a harmful Staphylococcus sp. infection or a condition caused thereby, comprising administering to a subject in need thereof an antimicrobial composition comprising Micrococcus porci, or a lysate, culture or extract thereof.

In this regard, the Micrococcus porci, or a lysate, culture or extract thereof, and harmful Staphylococcus sp. are the same as described above.

The condition of the subject may be a condition in need of antimicrobial treatment against harmful Staphylococcus sp.

As used herein, the terms “administering,” “applying,” “introducing,” and “implanting” are used interchangeably and may refer to the placement of a composition according to one embodiment of the invention into a subject by a method or route that results in at least partial localization of the composition to a desired site.

The composition of the present invention may be administered by any methods known in the art. The composition may be administered directly to the subject by any means, for example, by intravenous, intramuscular, oral, transdermal, mucosal, intranasal, intratracheal, or subcutaneous routes. The composition may be administered systemically or topically. For example, the antimicrobial composition can be applied to the skin.

The subject may be mammals, for example, humans, cattle, horses, pigs, dogs, sheep, goats, or cats. The subject may be an individual in need of antimicrobial treatment.

The composition according to one embodiment of the present invention may be administered to a subject in an amount of 0.00001 mg to 1,000 mg per day, for example, 0.00001 mg to 500 mg, 0.00001 mg to 100 mg, 0.00001 mg to 50 mg, 0.00001 mg to 25 mg, 1 mg to 1,000 mg, 1 mg to 500 mg, 1 mg to 100 mg, 1 mg to 50 mg, 1 mg to 25 mg, 5 mg to 1,000 mg, 5 mg to 500 mg, 5 mg to 100 mg, 5 mg to 50 mg, 5 mg to 25 mg, 10 mg to 1,000 mg, 10 mg to 500 mg, 10 mg to 100 mg, 10 mg to 50 mg, or 10 mg to 25 mg.

The dose may vary depending on factors such as formulation method, administration method, patient's age, weight, gender, pathological condition, food, administration time, administration route, excretion rate, and reaction sensitivity. A person skilled in the art can appropriately adjust the dose by taking these factors into consideration. The composition may be administered once a day or two or more times within the range of clinically acceptable side effects. The composition may be administered at one or more than two administration sites. The composition may be administered daily or at intervals of 2 to 5 days, and may be administered from 1 to 30 days per treatment. If necessary, the same treatment may be repeated after an appropriate period of time. For animals other than humans, the same dose per kg as for humans may be administered, or the above dose may be converted into, for example, the volume ratio (e.g., average value) of the organ (heart, etc.) between the target animal and human.

Another aspect of the present invention provides a method of inhibiting the growth of harmful Staphylococcus sp., comprising applying Micrococcus porci, or a lysate, culture or extract thereof to an area where the growth of harmful Staphylococcus sp. is suspected.

In this regard, the Micrococcus porci, or a lysate, culture or extract thereof, and the harmful Staphylococcus sp. are the same as described above.

The Micrococcus porci, or a lysate, culture or extract thereof may be applied to areas where growth of harmful Staphylococcus sp. is suspected according to conventional methods well known to those of ordinary skill in the art in various fields where it is necessary to inhibit the growth of harmful Staphylococcus sp. The Micrococcus porci, or a lysate, culture or extract thereof may be treated in an amount suitable for the intended use.

Another aspect of the present invention provides a selective antimicrobial method against harmful Staphylococcus sp., comprising treating a bacterium with Micrococcus porci, or a lysate, culture or extract thereof.

In this regard, the Micrococcus porci, or a lysate, culture or extract thereof, and their selective antimicrobial activity against harmful Staphylococcus sp. are the same as described above.

Bacteria may be appropriately treated with Micrococcus porci, or a lysate, culture or extract thereof according to conventional methods well known to those of ordinary skill in the art in a variety of fields where antimicrobials are required. The Micrococcus porci, or a lysate, culture or extract thereof may be treated in an amount suitable for the intended use.

Another aspect of the present invention provides a strain of Micrococcus porci deposited under Accession No. KACC 81219BP.

Said Micrococcus porci strain may comprise 16S rRNA of SEQ ID NO: 3.

Said strain may exhibit selective antimicrobial activity against harmful Staphylococcus sp. Specifically, the strain may exhibit antimicrobial activity against harmful Staphylococcus aureus but have weak or no effect on harmless skin beneficial bacteria.

Said Micrococcus porci strain may exhibit antimicrobial activity against harmful Staphylococcus sp., for example, skin disease-causing bacteria such as Staphylococcus aureus, Methicillin resistance Staphylococcus aureus (MRSA), and Staphylococcus capitis, among Staphylococcus genus bacteria (Staphylococcus aureus), but is not limited thereto. However, said Micrococcus porci strain has been found to have weak antimicrobial activity against Staphylococcus epidermidis, a beneficial skin bacteria, and no antimicrobial activity against Staphylococcus hominis, a beneficial skin bacteria. Furthermore, said Micrococcus porci strain has been found to exhibit no antimicrobial activity against Micrococcus luteus, Candida albicans SC5314, Corynebacterium propinguum, Pseudomonas aeruginosa ATCC10145, and Escherichia coli DH5a, which do not belong to Staphylococcus sp.

In one embodiment, said Micrococcus porci strain may show selective antimicrobial activity against one or more of Staphylococcus aureus, Methicillin resistance Staphylococcus aureus (MRSA), and Staphylococcus capitis.

In one embodiment, said Micrococcus porci strain may exhibit selective antimicrobial activity against Staphylococcus aureus, Methicillin resistance Staphylococcus aureus (MRSA), and Staphylococcus capitis.

In still another aspect, the present invention provides a lysate, culture or extract of said Micrococcus porci strain.

The Micrococcus porci strain, and a lysate, culture or extract thereof are the same as described above.

The lysate, culture or extract of said Micrococcus porci strain may exhibit selective antimicrobial activity against harmful Staphylococcus sp. In this regard, the selective antimicrobial activity against harmful Staphylococcus sp. is the same as described above for the Micrococcus porci strain.

### Effect of the Invention

A composition comprising Micrococcus porci, or a lysate, culture or extract thereof, as an active ingredient, according to one aspect of the present invention, has selective antimicrobial activity against harmful Staphylococcus sp., in particular Staphylococcus aureus, Methicillin resistance Staphylococcus aureus (MRSA), and Staphylococcus capitis. Therefore, the composition is useful as an antimicrobial composition for cosmetics, quasi-drugs, external skin preparations, medicines, etc.

### BRIEF DESCRIPTION OF DRAWINGS

FIG. 1 shows the growth inhibition of Staphylococcus aureus ATCC 25923, Methicillin resistance Staphylococcus aureus USA 300 (MRSA), Staphylococcus epidermidis ATCC 12228, Staphylococcus hominis, Staphylococcus capitis, Micrococcus luteus, Candida albicans SC5314, Corynebacterium propinguum, Pseudomonas aeruginosa ATCC10145, and Escherichia coli DH5a, when treated with a strain homogenate according to an embodiment of the present invention (N-CNT: negative control; Cell lysate: strain homogenate)

FIG. 2 is a graph showing the extent of biofilm formation of Staphylococcus aureus compared to a control group when treated with a strain homogenate according to one embodiment of the present invention (control group: PBS, 0% cell lysate; test group: lysate, 20% cell lysate).

FIG. 3 is a graph showing the cell viability of human keratinocytes when treated with a strain homogenate according to one embodiment of the invention (control group: 10% PBS; test group: 10% cell lysate).

FIG. 4 shows the nature of the active substances in the strain homogenate according to one embodiment of the present invention by measuring the antimicrobial activity of the strain homogenate against Staphylococcus aureus ATCC 25923 after heat treatment or treatment with protease K (heat treatment: 95° C. for 10 min, protease K; 1 mg/mL).

### DETAILED DESCRIPTION

Hereinafter, the present invention will be described in more detail with reference to examples. However, these examples are provided only for illustration of the present invention, and the scope of the present invention is not limited to these examples.

### Example 1. Strain Isolation and Identification

Samples were collected from the skin of healthy Korean individuals, mixed in phosphate buffered saline (PBS) solution, and inoculated into Luria-Berani (LB) medium by serial dilution. Bacterial colonies formed on a plate were selected after incubation at 34° C. for 63 hours, and purified by using streaking method under the same medium and conditions. The 16S rRNA gene was sequenced to confirm the molecular phylogenetic characterization of the cultured strains. Polymerase chain reaction (PCR) amplification was performed for 32 cycles at 95° C. for 30 seconds, 55° C. for 30 seconds, 72° C. for 1 minute 45 seconds, and finally 72° C. for 5 minutes. At this time, primers (SEQ ID NOs: 1 and 2) designed to specifically react with common bacterial genes were used. The PCR amplification products were purified and sequenced (Macrogen, Korea), and the sequences were analyzed using the BLAST program of the National Center for Biotechnology Information (NCBI). When compared with other previously registered strains, species with more than 99% base sequence homology were determined to be related species. The strain isolated according to this example has a 16S rRNA sequence with SEQ ID NOs: 3 (complementary DNA) and was identified as belonging to the genus Micrococcus sp. The inventors deposited the above strain with the Agricultural Genetic Resource Center on May 24, 2022, under Accession No. KACC 81219BP, and it was finally identified as Micrococcus porci and named Micrococcus porci CBN008.

TABLE 1

| | | SEQ ID |
|---|---|---|
| Description | Sequence | NO: |
| primer | AGAGTTTGATCCTGGCTCAG | 1 |
| primer | TACGGTTACCTTGTTACGACTT | 2 |
| 16S rRNA | GCTCAGGATGAACGCTGGCGGCGTGOTTAACACATGCAA | 3 |
| | GTCGAACGATGAAGCCCAGCTTGCTGGGTGGATTAGTGG | |
| | CGAACGGGTGAGTAACACGTGAGTAACCTGCCCTTGACT | |
| | CTGGGATAAGCCTGGGAAACTGGGTCTAATACCGGATAG | |
| | GAACGTCCACCGCATGGTGGGTGTTGGAAAGAATTTCGG | |
| | TCATGGATGGACTCGCGGCCTATCAGCTTGTTGGTGAGG | |
| | TAATGGCTCACCAAGGCGACGACGGGTAGCCGGCCTGAG | |
| | AGGGTGACCGGCCACACTGGGACTGAGACACGGCCCAG | |
| | ACTCCTACGGGAGGCAGCAGTGGGGAATATTGCACAATG | |
| | GGCGAAAGCCTGATGCAGCGACGCCGCGTGAGGGATGA | |
| | CGGCCTTCGGGTTGTAAACCTCTTTCAGTAGGGAAGAAGC | |
| | GAAAGTGACGGTACCTGCAGAAGAAGCACCGGCTAACTA | |
| | CGTGCCAGCAGCCGCGGTAATACGTAGGGTGCGAGCGTT | |
| | ATCCGGAATTATTGGGCGTAAAGAGCTCGTAGGCGGTTTG | |
| | TCGCGTCTGTCGTGAAAGTCCGGGGCTTAACCCCGGATC | |
| | TGCGGTGGGTACGGGCAGACTAGAGTGCAGTAGGGGAG | |
| | ACTGGAATTCCTGGTGTAGCGGTGGAATGCGCAGATATCA | |
| | GGAGGAACACCGATGGCGAAGGCAGGTCTCTGGGCTGTA | |
| | ACTGACGCTGAGGAGCGAAAGCATGGGGAGCGAACAGG | |
| | ATTAGATACCCTGGTAGTCCATGCCGTAAACGTTGGGCAC | |
| | TAGGTGTGGGGACCATTCCACGGTTTCCGCGCCGCAGCT | |
| | AACGCATTAAGTGCCCCGCCTGGGGAGTACGGCCGCAAG | |
| | GCTAAAACTCAAAGGAATTGACGGGGGCCCGCACAAGCG | |
| | GCGGAGCATGCGGATTAATTCGATGCAACGCGAAGAACC | |
| | TTACCAAGGCTTGACATGTTTTCGACCGCCGTAGAGATAC | |
| | GGTTTCCCCTTTGGGGCGGATTCACAGGTGGTGCATGGT | |
| | TGTCGTCAGCTCGTGTCGTGAGATGTTGGGTTAAGTCCCG | |
| | CAACGAGCGCAACCCTCGTTCCATGTTGCCAGCACGTAG | |
| | TGGTGGGGACTCATGGGAGACTGCCGGGGTCAACTCGGA | |
| | GGAAGGTGGGGACGACGTCAAATCATCATGCCCCTTATG | |
| | TCTTGGGCTTCACGCATGCTACAATGGCCGGTACAATGG | |
| | GTTGCGATACTGTGAGGTGGAGCTAATCCCAAAAAGCCG | |
| | GTCTCAGTTCGGATTGGGGTCTGCAACTCGACCCCATGAA | |
| | GTCGGAGTCGCTAGTAATCGCAGATCAGCAACGCTGCGG | |
| | TGAATACGTTCCCGGGCCTTGTACACACCGCCCGTCAAGT | |
| | CACGAAAGTCGGTAACACCCGAAGCCGGTGGCCTAACCC | |
| | TT | |

### Example 2. Cultivation and Homogenization of Strains

The strain isolated in Example 1 was cultured in Mueller Hinton (MH) broth medium in an incubator at 30° C. and cells were collected at the end of the exponential phase of the growth curve. The bacterial cells were centrifuged at 8,000 rpm for 30 minutes and washed once with phosphate-buffered saline (PBS). The cells were dissolved in PBS and homogenized using a high-pressure homogenizer. The homogenate was filtered through a 0.2 μm filter to remove bacterial cells and quantified to 2 mg/ml using the Bradford assay for further evaluation and activity analysis. Evaluation and activity analysis were performed using a strain homogenate that had been manufactured for no more than 1 day.

### Experimental Example 1. Antimicrobial Activity

The strain homogenate obtained in Example 2 was assayed for antimicrobial activity against harmful strains.

Specifically, antimicrobial activity was determined against Staphylococcus aureus ATCC 25923, Methicillin resistance Staphylococcus aureus USA 300 (MRSA), Staphylococcus epidermidis ATCC 12228, Staphylococcus hominis, Staphylococcus capitis, Micrococcus luteus, Candida albicans SC5314, Corynebacterium propinquum, Pseudomonas aeruginosa ATCC10145, and Escherichia coli DH5a. First, the strains were cultured in TSB or MH medium to be used in experiments. Bacteria were grown in an aerobic chamber at 34° C., and yeast was grown in an aerobic chamber at 30° C. The cultures were subcultured to 1/100 (seed/media) at the end of the exponential phase according to the growth curve. The cell lines that reached the beginning of the exponential phase of the growth curve were evaluated for antimicrobial activity by preparing dilutions with an OD600 of 0.01.

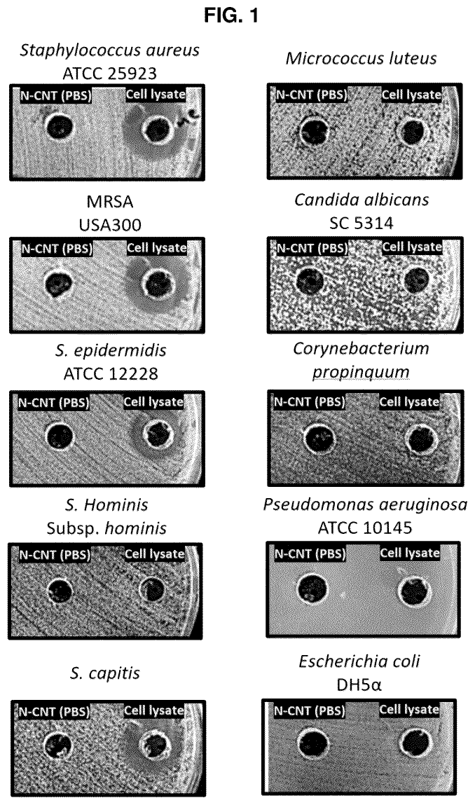

The antimicrobial activity of the above strain homogenate was determined under the following conditions. First, dilutions of the above 10 strains (S. aureus, MRSA, S. epidermidis, S. hominis, S. capitis, M. luteus, C. albicans, C. propinquum, P. aeruginosa, and E. coli) to be evaluated for antimicrobial activity were spread twice on MH agar plates using sterilized cotton swabs to form agar pits with a diameter of 0.8 cm. The agar pits were inoculated with 150 μL of the strain homogenate prepared in Example 2, and then grown in an incubator at 30° C. for 24 and 48 hours to observe the clear zone produced, the results of which are shown in FIG. 1.

FIG. 1 shows the growth inhibition of Staphylococcus aureus ATCC 25923, Methicillin resistance Staphylococcus aureus USA 300 (MRSA), Staphylococcus epidermidis ATCC 12228, Staphylococcus hominis, Staphylococcus capitis, Micrococcus luteus, Candida albicans SC5314, Corynebacterium propinguum, Pseudomonas aeruginosa ATCC10145, and Escherichia coli DH5a, when treated with a strain homogenate according to an embodiment of the present invention (N-CNT: negative control; Cell lysate: strain homogenate)

As shown in FIG. 1, clear zones were formed for S. aureus ATCC 25923, MRSA, S. epidermidis, and S. capitis treated with the strain homogenate of Example 2. Specifically, clear zones were formed for skin pathogens such as S. aureus ATCC 25923, MRSA, and S. capitis, which indicates that the strain homogenate of Example 2 has antimicrobial activity. In contrast, the clear zone was reduced and faint for a skin beneficial bacterium such as S. epidermidis, compared to the skin pathogens such as S. aureus ATCC 25923, MRSA, and S. capitis. This indicates that the strain homogenate of Example 2 showed relatively weak antimicrobial activity against skin beneficial bacteria. In Staphylococcus hominis, a skin beneficial bacterium, no clear zone was found to form, indicating little antimicrobial activity.

In addition, Micrococcus luteus, Candida albicans SC5314, Corynebacterium propinguum, Pseudomonas aeruginosa ATCC10145, and Escherichia coli DH5a, which do not belong to Staphylococcus sp. showed little clear zone formation upon treatment with the strain homogenate of Example 2, indicating little antimicrobial activity. These results demonstrate that the strain homogenate of Example 2 exhibits selective antimicrobial activity against harmful Staphylococcus sp., particularly Staphylococcus aureus, Methicillin resistance Staphylococcus aureus (MRSA), and Staphylococcus capitis.

### Experimental Example 2. Ability to Inhibit Biofilm Formation

The present experiment was performed to determine whether the strain homogenate obtained in Example 2 inhibits the biofilm-forming ability of Staphylococcus aureus ATCC 25923.

Specifically, frozen stocks of S. aureus strains were first inoculated into MH medium and incubated at 34° C. for 17 hours. The strain was then inoculated again into fresh MH medium and grown to reach the beginning of the exponential phase of the growth curve at 30° C. in a serial culture.

Prior to the experiment, 160 μL of MH broth supplemented with 1% glucose was added to all available wells of a 96-well plate. Half of the available wells were treated with 40 μL of PBS, and the other half of the available wells were treated with 40 ul of cell homogenate at a protein concentration of 2 mg/ml. Then, 2 μL of S. aureus lineage culture was added to all wells and grown for 24 hours at 30° C. and 200 rpm. PBS-treated wells were used as negative controls.

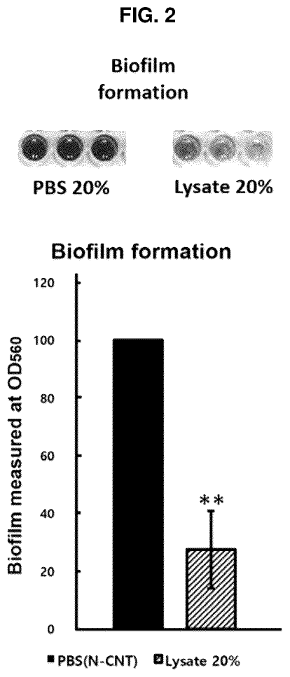

The wells were then washed twice with distilled water, dried to fix the biofilm, and the wells were stained with a 0.1% crystal violet solution. The wells were then washed three times with distilled water and the biofilm was dissolved with 30% acetic acid solution. The absorbance at 560 nm was measured using a microplate reader, and the results are shown in FIG. 2.

FIG. 2 is a graph showing the extent of biofilm formation of Staphylococcus aureus when treated with the strain homogenate of Example 2, by measuring the absorbance of Staphyylococcus aureus according to the WST-8 assay (control group: PBS, 0% cell lysate; test group: lysate, 20% cell lysate).

As shown in FIG. 2, S. aureus treated with the strain homogenate reduced the biofilm production to 27.5% compared to the control group treated with PBS. It is verified that the strain homogenate can significantly inhibit the biofilm formation of S. aureus.

### Experimental Example 3. Evaluation of Relative Cell Viability

Cell viability was tested to determine whether the strain isolated in Example 1 is toxic to cells.

Specifically, a human epidermal keratinocyte cell line (HaCaT) was first inoculated into 24-well plates at a density of 2.5×10⁴ cells/well in DMEM medium (Dubelcco's modified eagle medium) containing 10% fetal bovine serum (FBS) and incubated overnight, followed by incubation for 24 hours in DMEM medium without FBS.

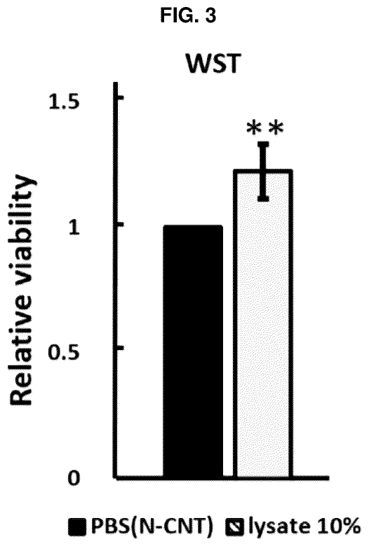

The cytotoxicity of the strain homogenate was evaluated by the WST assay. The cultured cell lines were treated with the strain homogenate (2 mg/ml protein concentration) or PBS at 10% concentration of the culture medium and then incubated under the same conditions for 24 hours. At this time, the 10% PBS treatment group was used as a negative control, with a total volume of 500 μL for each well. After removing the culture medium, the cells were washed once with 1×PBS, and cultured at 37° C. for 0.5 to 4 hours after adding DMEM medium containing WST-8 reagent. The absorbance at 450/625 nm was then measured using a microplate reader, and the results are shown in FIG. 3.

FIG. 3 is a graph showing the cell viability of human keratinocytes when treated with a strain homogenate according to one embodiment of the invention, as measured by WST assay (control group: 10% PBS; test group: 10% cell lysate).

As shown in FIG. 3, treatment with 10% (v/v) of the strain homogenate showed no significant difference in the viability of human keratinocytes compared to the negative control treated with PBS, indicating that the strain homogenate is not cytotoxic. These results indicate that the homogenate of the above strain is safe for application to the skin.

### Experimental Example 4. Exploration on Antibiotics

It was observed whether changes in antimicrobial activity against S. aureus ATCC 25923 occurred when the strain homogenate obtained in Example 2 was subjected to heat treatment and/or treated with a proteolytic enzyme, Protease K.

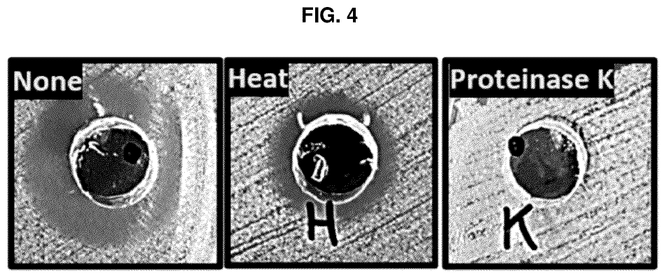

Specifically, as previously shown in Example 1, the strain homogenate of Example 2 exhibits antimicrobial activity against Staphylococcus aureus, Methicillin resistance Staphylococcus aureus (MRSA), and Staphylococcus capitis. This experiment was conducted to determine what substance(s) may be responsible for this activity. The same experimental method was used as in Experimental Example 1. S. aureus ATCC 25923 was used as an antimicrobial target strain. The strain homogenate was prepared as in Example 2, then aliquoted into 1.5 mL portions and divided into three E-tubes, each of which was subjected to subsequent processing. One tube was kept intact in an ice bucket, one tube was treated at 95° C. for 10 minutes, and one tube was treated with protease K at a concentration of 1 mg/mL. After each treatment, 150 μL of the strain homogenate was inoculated into agar pits and cultured in an incubator at 30° C. for 24 and 48 hours to observe the formation of clear zone, and the results are shown in FIG. 4.

FIG. 4 shows the results of an experiment to determine the properties of the active substance in the strain homogenate by measuring the antimicrobial activity of the strain homogenate against Staphylococcus aureus ATCC 25923 after heat treatment or treatment with protease K (heat treatment: 95° C. for 10 min, protease K; 1 mg/mL).

As shown in FIG. 4, heat treatment of the strain homogenate or treatment with protease K reduced the clear zone, indicating that the antimicrobial activity was inhibited. In particular, in the case of protease K treatment, the clear zone disappeared completely. In light of this, it is assumed that the antimicrobial activity observed in the strain homogenate is caused by a protein.

Accession No.

Depositary Authority: Korea Agricultural Research Center Microbiology Bank (KACC)

Accession No.: KACC81219BP

Accession Date: May 24, 2022

What is claimed is:

1. A method of treating, ameliorating, or alleviating a harmful Staphylococcus sp. infection or a condition caused thereby, comprising:
administering to a subject in need thereof an antimicrobial composition comprising Micrococcus porci, or a lysate, culture or extract thereof.

2. The method of claim 1, wherein the antimicrobial composition exhibits selective antimicrobial activity only against harmful Staphylococcus sp., with weak or no antimicrobial activity against harmless skin beneficial bacteria.

3. The method of claim 2, wherein the harmful Staphylococcus sp. is one or more microorganisms selected from the group consisting of Staphylococcus aureus, Methicillin resistance Staphylococcus aureus (MRSA), and Staphylococcus capitis.

4. The method of claim 1, wherein the Micrococcus porci is a strain of Micrococcus porci deposited under Accession No. KACC 81219BP.

5. The method of claim 1, wherein said antimicrobial composition is a pharmaceutical composition, a nonpharmaceutical composition, a cosmetic product, or a food product.

6. The method of claim 1, wherein said antimicrobial composition further comprises a carrier, an excipient, a diluent, an additive, or a combination thereof.

7. A method of inhibiting the growth of harmful Staphylococcus sp., comprising:
applying an antimicrobial composition comprising Micrococcus porci, or a lysate, culture or extract thereof, to an area where the growth of harmful Staphylococcus sp. is suspected.

8. The method of claim 7, wherein the antimicrobial composition exhibits selective antimicrobial activity only against harmful Staphylococcus sp., with weak or no antimicrobial activity against harmless skin beneficial bacteria.

9. The method of claim 8, wherein said harmful Staphylococcus sp. is one or more microorganisms selected from the group consisting of Staphylococcus aureus, Methicillin resistance Staphylococcus aureus (MRSA), and Staphylococcus capitis.

10. The method of claim 7, wherein the Micrococcus porci is a strain of Micrococcus porci deposited under Accession No. KACC 81219BP.

11. The method of claim 7, wherein the antimicrobial composition is a pharmaceutical composition, a quasi-drug composition, a cosmetic, or a food product.

12. The method of claim 7, wherein said antimicrobial composition further comprises a carrier, an excipient, a diluent, an additive, or a combination thereof.

13. A strain of Micrococcus porci deposited under Accession No. KACC 81219BP.

14. A lysate of the strain according to claim 13.

15. A culture of the strain according to claim 13.

16. An extract of the strain according to claim 13.


SEQUENCE LISTING (3 sequences)

SEQ ID NO: 1 - DNA, 20 bp, synthetic construct
  source 1..20: mol_type = other DNA
agagtttgatcctggctcag

SEQ ID NO: 2 - DNA, 22 bp, synthetic construct
  source 1..22: mol_type = other DNA
tacggttaccttgttacgactt

SEQ ID NO: 3 - DNA, 1410 bp, synthetic construct
  source 1..1410: mol_type = other DNA
gctcaggatgaacgctggcggcgtgcttaacacatgcaagtcgaacgatgaagcccagcttgctgggtggattagtggcgaacgggtgagtaacacgtgagtaacctgcccttgactctgggataagcctgggaaactgggtctaataccggataggaacgtccaccgcatggtgggtgttggaaagaatttcggtcatggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttttcgaccgccgtagagatacggtttcccctttggggcggattcacaggtggtgcatggttgtcgtcagctcgtgtcgtgagatgttgggttaagtcccgcaacgagcgcaaccctcgttccatgttgccagcacgtagtggtggggactcatgggagactgccggggtcaactcggaggaaggtggggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcctaaccctt